(19) United States
(12) Patent Application Publication
(10) Pub. No.: US 2008/0199861 A1
(43) Pub. Date: Aug. 21, 2008

(54) Real-time microarray apparatus and methods related thereto

(75) Inventors: Yuandong Gu, Plymouth, MN (US); Leon Xu, Shanghai (CN)

Correspondence Address: HONEYWELL INTERNATIONAL INC., 101 COLUMBIA ROAD, P O BOX 2245, MORRISTOWN, NJ, 07962-2245, US

(73) Assignee: Honeywell International, Inc.

(21) Appl. No.: 11/706,448

(22) Filed: Feb. 15, 2007

(51) Int. Cl. C12Q 1/68; C12M 1/00

(52) U.S. Cl. 435/6; 435/287.1

(57) ABSTRACT

Embodiments of the invention relate to a real-time microarray apparatus comprising an upper substrate, a lower substrate, a buffer positioned between the upper and lower substrate, a microarray positioned on either the upper substrate or the lower substrate, a heater positioned near the microarray, a pump positioned near the buffer and microarray and an imaging sensor positioned near the microarray.

### TECHNICAL FIELD

Embodiments of the present invention relate to a real-time microarray apparatus. More specifically, embodiments of the present invention relate to a real-time PCR microarray apparatus.

### BACKGROUND

Active microarrays that integrate nucleic acid sample preparation, on-chip nucleic acid amplification and microarrays are highly desired for their ease of use, varied functionality and other advantages. Active microarrays currently used employ a nucleic acid amplification process first, such as polymerase chain reaction (PCR), and then proceed with microarray hybridization. When a target nucleic acid, such as DNA, is at a very low concentration, the under-amplified DNA may not give a strong enough signal for microarray detection. In this situation, retesting will result which may consume excess time and resources.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates a cross-sectional view of a real-time microarray apparatus 100, according to some embodiments.

FIG. 2 illustrates a schematic of a real-time microarray apparatus 200, according to some embodiments.

FIG. 3 illustrates a block flow diagram of a method of detecting a target nucleic acid 300, according to some embodiments.

### SUMMARY

Embodiments of the invention relate to a real-time microarray apparatus comprising an upper substrate, a lower substrate, a buffer positioned between the upper and lower substrate, a microarray positioned on either the upper substrate or the lower substrate, a heater positioned near the microarray, a pump positioned near the buffer and microarray and an imaging sensor positioned near the microarray.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Embodiments of the invention relate to a real-time microarray apparatus utilizing a heater to release under amplified nucleic acid samples on a microarray to be re-amplified and further detected. The methods and apparatus of the present invention reduce the amount of retesting of low concentration nucleic acid on a microarray. A microarray is a collection of microscopic nucleic acid (e.g., DNA) spots attached to a solid surface, forming an array for the purpose of expression profiling (monitoring expression levels for thousands of genes simultaneously). The combined nucleic acid amplification and hybridization reaction can be utilized with microarray applications for optimal analysis.

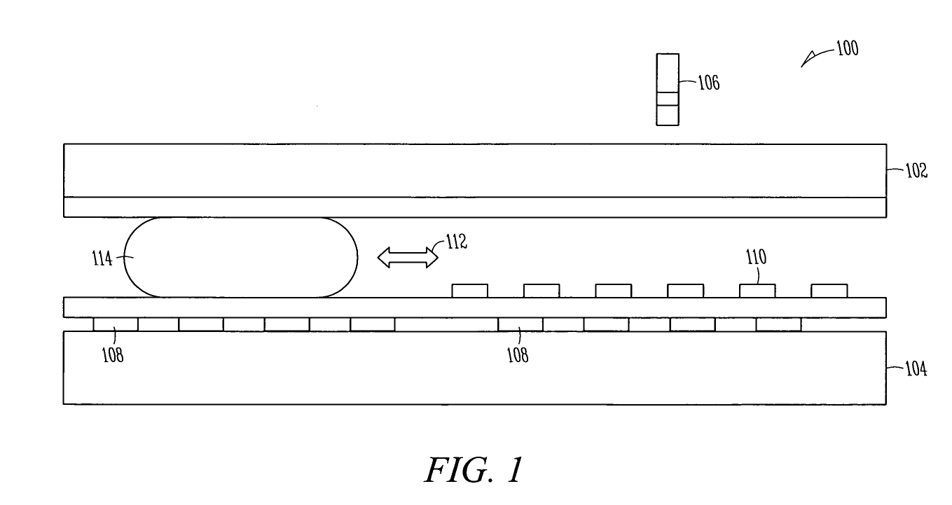

Referring to FIG. 1, a cross-sectional view of a real-time microarray apparatus 100 is shown, according to some embodiments. The apparatus 100 includes an upper substrate 102 and a lower substrate 104, which may form or be part of a reaction chamber. An amplification region 114 may support a nucleic acid amplification process, such as polymerase chain reaction (PCR), in a buffer. A nucleic acid sample may by hybridized on a microarray 110. The buffer may support both the amplification process and hybridization process. The arrow 112 may represent a partition between the amplification region 114 and the microarray 110 or may indicate a nucleic acid movement mechanism, such as a pump, for example. An imaging sensor 106 may be positioned near the microarray 110. The imaging sensor 106 may be a CCD camera, for example. A heater 108 may be positioned near the microarray 110. The heater 108 may also be positioned near the amplification region, for example. Movement of the nucleic acid sample within the reaction chamber or between the amplification region 114 and microarray 110 may be accomplished by utilization of a pump, such as a pneumatic pump, step motor pump or electrostatic pump, for example. Movement of the sample may also be realized by utilizing electrowetting or optoelectrowetting, for example.

The heater 108 may be comprised of multiple heaters or multiple parts of heaters capable of maintaining separate temperatures. For example, a heater may be positioned to denature under-amplified nucleic acid at one temperature (for example, about 90° C. and above), a separate heater or portion of a single heater may be heated to a lower temperature to anneal or hybridize (such as about 60° C.) in a separate portion of the reaction chamber.

The substrate (upper 102 and lower 104) may be comprised of an optical material, such as glass or polymer, for example. The nucleic acid sample, or target nucleic acid, may be DNA, RNA or both, for example. The heater 108 may be a wire, heat plate or utilize hot air or radiant heat for example. The heater 108 may be positioned within the reaction chamber, integrated within the structure of the reaction chamber or positioned on the outside of the reaction chamber, for example.

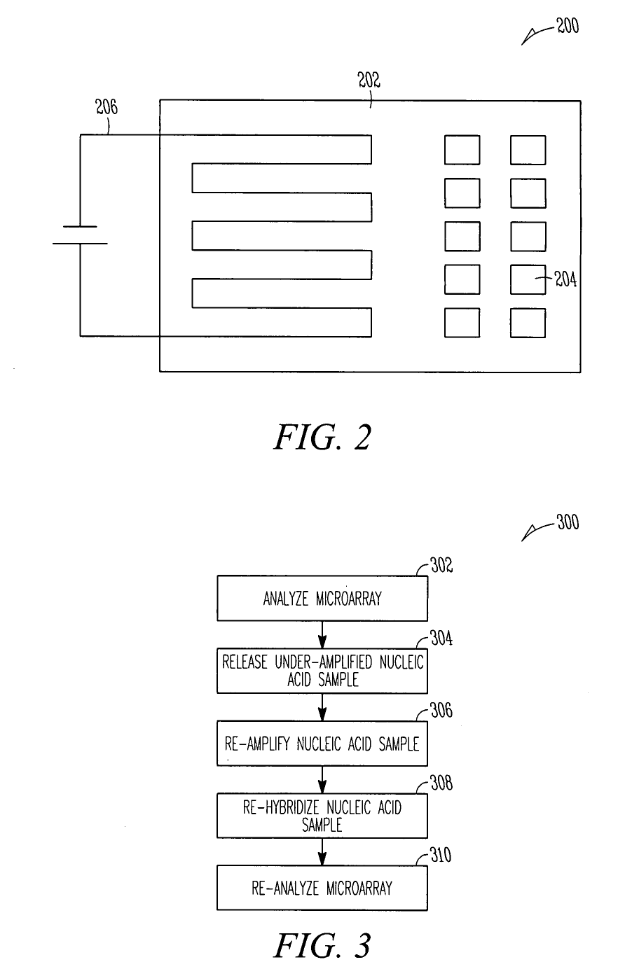

Referring to FIG. 2, a schematic of a real-time microarray apparatus 200 is shown, according to some embodiments. A substrate 202 may support a microarray 204, for example. A heater 206 may be positioned near the microarray 204. The heater 206 may be positioned under the microarray 204 or near the microarray at a sufficient distance to affect the hybridization temperature, for example.

Referring to FIG. 3, a block flow diagram of a method of detecting a target nucleic acid 300 is shown, according to some embodiments. A microarray may be analyzed 302, sufficient to identify any under-amplified nucleic acid sample. Analyzing 302 may be accomplished by evaluating the signal produced by contacting the microarray with light, for example. A fluorescent signal may be generated when contacting the microarray with an evanescent wave, for example. The under-amplified nucleic acid sample may be released 304 by activating the heater. The nucleic acid sample may be released 304 by denaturing at the annealing temperature, such as about 90° C. or above, for example. The nucleic acid sample may be re-amplified 306 in the buffer and then re-hybridized 308 on the microarray. The microarray may then be re-analyzed 310.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed is:

1. A real-time microarray apparatus, comprising:
an upper substrate;
a lower substrate;
a buffer, positioned between the upper and lower substrate;
a microarray, positioned on either the upper substrate or the lower substrate;
a heater, positioned near the microarray; and
an imaging sensor, positioned near the microarray.

2. The microarray of claim 1, wherein the upper substrate and lower substrate form a reaction chamber.

3. The microarray of claim 1, wherein the upper substrate and lower substrate are part of a reaction chamber.

4. The microarray of claim 1, further comprising a pump positioned near the microarray.

5. The microarray of claim 1, wherein the heater comprises a wire.

6. The microarray of claim 1, wherein the heater comprises a heated plate.

7. The microarray of claim 1, wherein the upper and lower substrate comprise glass.

8. The microarray of claim 1, wherein the upper and lower substrate comprise polymer.

9. The microarray of claim 1, wherein the imaging sensor comprises a CCD camera.

10. The microarray of claim 4, wherein the pump comprises a pneumatic pump.

11. The microarray of claim 4, wherein the pump comprises a step motor pump.

12. The microarray of claim 4, wherein the pump comprises an electrostatic pump.

13. The microarray of claim 1, wherein the buffer is capable of supporting a nucleic acid amplification process and hybridization process.

14. A method of detecting a nucleic acid sample, the method comprising:
analyzing a microarray;
releasing under-amplified nucleic acid sample;
re-amplifying the nucleic acid sample;
re-hybridizing the nucleic acid sample; and
re-analyzing the microarray.

15. The method of claim 14, wherein analyzing comprises evaluating the fluorescent signal of a nucleic acid sample on a microarray.

16. The method of claim 14, wherein releasing comprises heating a hybridized nucleic acid sample to an annealing temperature sufficient to denature it.

17. The method of claim 14, wherein re-amplifying the nucleic acid sample comprises undergoing polymerase chain reaction (PCR).

18. The method of claim 14, wherein between the steps of releasing and re-amplifying, the step of pumping the nucleic acid sample from the microarray to an amplification region.

19. The method of claim 14, wherein between the steps of re-amplifying and re-hybridizing, the step of pumping the nucleic acid sample from the amplification region to the microarray.
